(19) United States
(12) Patent Application Publication
(10) Pub. No.: US 2012/0315671 A1
(43) Pub. Date: Dec. 13, 2012

(54) EXPRESSION MEDIA FOR PROTEINS IN YEAST SYSTEM

(75) Inventors: NITIN JAIN, Knoxville, TN (US); Seth Andrew Albright, Maryville, TN (US)

(73) Assignee: UNIVERSITY OF TENNESSEE RESEARCH FOUNDATION, Knoxville, TN (US)

(21) Appl. No.: 13/492,969

(22) Filed: Jun. 11, 2012

Related U.S. Application Data
(60) Provisional application No. 61/496,217, filed on Jun. 13, 2011.

(51) Int. Cl. C12N 1/16; C12N 9/00; C12P 21/00

(52) U.S. Cl. 435/69.3; 435/255.7; 435/255.5; 435/69.1; 435/69.6; 435/69.4; 435/183; 435/69.7

(57) ABSTRACT

The subject invention provides advantageous new media formulations, methods for their production, methods for cultivating cells using the media as well as compositions thereof and their use for enhanced expression of recombinant proteins. In certain embodiments, the subject invention provides media for use in producing recombinant proteins in yeast systems, such as Pichia pastoris.

### CROSS-REFERENCE TO RELATED APPLICATION

This application claims the benefit of U.S. Provisional Application Ser. No. 61/496,217, filed Jun. 13, 2011, the disclosure of which is hereby incorporated by reference in its entirety, including all figures, tables and amino acid or nucleic acid sequences.

### GOVERNMENT SUPPORT

This invention was made with government support under Contract No. 1 R01 AI060818-01 awarded by National Institutes of Health. The government has certain rights in the invention.

### BACKGROUND OF THE INVENTION

Cell culture media provide the nutrients necessary to maintain and grow cells in a controlled, artificial and in vitro environment. Characteristics and compositions of the cell culture media vary depending on the particular cellular requirements. Important parameters include osmolality, pH, and nutrient formulations.

Media formulations have been used to cultivate a number of cell types including animal, plant and bacterial cells. Cells cultivated in culture media catabolize available nutrients and produce useful biological substances such as monoclonal antibodies, hormones, growth factors, viruses and the like. Such products have therapeutic applications and, with the advent of recombinant DNA technology, cells can be engineered to produce large quantities of these products. Thus, the ability to cultivate cells in vitro is not only important for the study of cell physiology, but is also necessary for the production of useful substances which may not otherwise be obtained by cost-effective means.

Methylotrophic yeasts, such as Hansenula polymorphs and Pichia pastoris, have been used as hosts for heterologous gene expression. Cregg et al., Bio/Technology, 5:479-485 (1987); Tschopp et al., Bio/Technology, 5:1305-1308 (1987) and for use in the production of heterologous proteins.

Given the recognized utility of methylotrophic yeast, as well as other yeast, in the expression of heterologous proteins, it is of interest to provide cell culture media that maintain and optimize cell growth and expression in a controlled, artificial and in vitro environment. There exists a current need for such media, particularly for isotopic labeling.

### BRIEF SUMMARY OF THE INVENTION

The subject invention provides advantageous new media formulations, methods for their production, methods for cultivating cells using the media as well as compositions thereof and their use for enhanced expression of recombinant molecules/proteins. In certain embodiments, the subject invention provides media for use in producing recombinant proteins in yeast systems, such as Pichia pastoris.

### BRIEF DESCRIPTION OF THE DRAWINGS

FIG. 1. Comparison of expression of 3 different proteins in Pichia Pastoris with various media. Three different proteins, Interleukin-8 (IL-8), Human Serum Albumin (HSA) and glycoprotein CD14 were secreted in 50 mL shake-flask cultures of rich BMMY media and formulation media of the subject invention under identical conditions. SDS-PAGE gels were run on each secreted sample without purification. The bands for the respective proteins are marked with arrows on the gel. Molecular weights of the protein are: IL-8 (9 kD), CD14 (32 kD) and HSA (68 kD). Lane 1: IL-8 in BMMY media. Lane 2: IL-8 in formulation media of the subject invention. Lane 3: HSA in BMMY media. Lane 4: HSA in formulation media of the subject invention. Lane 5: CD14 in BMMY media. Lane 6: CD14 in formulation media of the subject invention.

FIG. 2. 15N-HSQC spectrum of purified IL-8 with 15N labeling in new media.

### DETAILED DISCLOSURE OF THE INVENTION

A common issue associated with the production of recombinant proteins using yeast based expression systems is the presence of a multitude of extraneous proteins within the culture medium into which a recombinant protein is expressed. Thus, one aspect of the instant invention provides a new formulation for a medium that can be used for enhanced expression of proteins in yeast systems, such as Pichia pastoris.

One aspect of the invention provides a medium that enables complete isotopic labeling of proteins for the purpose of NMR studies. The medium of the invention has been tested on several different proteins and has been found to outperform commercial media currently available for isotopic labeling. Additional attributes of the subject medium include ease and cost-effectiveness in preparation compared to other commercial media for isotopic labeling and longer-term stability for shipping as well as storage due to preparation in powder form.

By “cell culture” or “culture” is meant the maintenance of cells in an artificial, e.g., an in vitro environment.

By “cultivation” is meant the maintenance of cells in an artificial environment under conditions favoring growth, differentiation or continued viability, in an active or quiescent state, of the cells. Thus, “cultivation” may be used interchangeably with “culture” or any of its synonyms described above.

By “culture vessel” is meant a glass, plastic, or metal container that can provide an aseptic environment for culturing cells.

The phrases “cell culture medium,” “culture medium” (plural “media” in each case) and “medium formulation” refer to a nutritive solution that supports the cultivation and/or growth of cells; these phrases may be used interchangeably.

The term “contacting” refers to the placing of cells to be cultivated into a culture vessel with the medium in which the cells are to be cultivated. The term “contacting” encompasses mixing cells with medium, pipetting medium onto cells in a culture vessel, and submerging cells in culture medium.

The present invention provides culture media and methods for their production. Culture media produced by the present methods are any media that may be used to support the growth of a cell, particularly a yeast cell. Culture media are produced in liquid form or in powdered form.

A culture medium of the invention is composed of a number of ingredients. In one embodiment, ingredients suitable for preparing a culture medium of the invention include those listed in Table 1.

TABLE 1

| Reagent | Grade | Manufacturer | Vendor |
|---|---|---|---|
| Water | cell culture | Hyclone | Fisher |
| adenine | 99% | Alfa Aesar | Fisher |
| guanosine | 99% | Acros Organic | Fisher |
| thymine | 99% | Acros Organic | Fisher |
| uracil | 99% | Acros Organic | Fisher |
| cytosine | 99% | Acros Organic | Fisher |
| sodium acetate | USP/FCC/EP/BP | Fisher | Fisher |
| succinic acid | anhydrous | Sigma | |
| KH2PO4 | HPLC grade, ACS | Fisher | Fisher |
| | cert. | | |
| K2HPO4 | USP grade | Fisher | Fisher |
| KOH | ACS certified | Fisher | Fisher |
| Yeast Nitrogen Base | n/a | Difco | Fisher |
| (YNB) w/o amino acids | | | |
| and ammonium sulfate | | | |
| methanol | optima, ACS specs. | Fisher | Fisher |
| dextrose | ACS certified | Fisher | Fisher |
| ammonium sulfate | ACS certified | Fisher | Fisher |
| MgSO4•7H2O | ACS specs. | Fisher | Fisher |
| FeCl3•6H2O | ACS specs. | J. T. Baker | |
| CaCl2•2H2O | Rnase, Dnase, | Fisher | Fisher |
| | Protease free | | |
| ZnSO4•7H2O | ACS certified | J. T. Baker | |
| MnSO4•H2O | ACS reagent grade | Acros Organic | Fisher |
| L-tryptophan | sigma grade | Sigma | |
| thiamine (vitamin B1) | n/a | Sigma Aldrich | |
| nicotinic acid (niacin) | 99.5% | Acros Organic | Fisher |
| biotin | 99% | Acros Organic | Fisher |

Another aspect of the invention provides a medium comprising succinic acid or succinate (or combinations thereof) in combination with sodium acetate, potassium acetate or acetic acid (or combinations thereof). In various, embodiments of this aspect of the invention, the medium can further comprise one or more of the following components: adenine, guanosine, thymine, uracil, cytosine, KH₂PO₄, K₂HPO₄, KOH, 10× YNB, an antibiotic, such as ampicillin and metal/vitamin solution. Thus, various embodiments of the invention provide a medium comprising:

a) succinate and/or succinic acid;

b) a source of acetate selected from: 1) sodium acetate; 2) potassium acetate; 3) acetic acid; 4) sodium acetate and potassium acetate; 5) sodium acetate and acetic acid; 6) potassium acetate and acetic acid; or 6) sodium acetate, potassium acetate and acetic acid; and, optionally,

c) one or more of the following components: adenine, guanosine, thymine, uracil, cytosine, KH₂PO₄, K₂HPO₄, KOH, 10× YNB, an antibiotic, such as ampicillin and/or metal/vitamin solution/mixture as disclosed herein. In some embodiments, the medium can have pyruvate, oxaloacetate or alphaketoglutarate, or any combination thereof, substituted for the succinate and/or succinic acid listed in subpart (a). Other embodiments provide for any combination of pyruvate, oxaloacetate, alphaketoglutarate, succinate and/or succinic acid to be used as the medium component disclosed in subpart (a), provided that the combination of materials is added in an amount substantially equal to the amount of succinate and/or succinic acid added to a medium as described in Table 2, 3 or 4.

According to the subject invention, methods for preparing nutritive medium for culturing cells and the expression of recombinant proteins, including isotopic labeling of the expressed proteins, comprise: in an optionally sterilized container or culture vessel (such as a bottle) one adds the following pre-sterilized components—10.88 g KH₂PO₄, 2.12 g K₂HPO₄, and 0.68 g of KOH. Next add 4.5 g sodium acetate (trihydrate), 1.5 g adenine, 1.95 g guanosine, 0.60 g thymine, 1.5 g uracil, 0.60 g cytosine, 1.5 g succinic acid, 2.6 gms Yeast nitrogen base and a trace metal mix (25 mg MgSO₄-7H₂O, 28 mg FeCl₃-6H₂O, 2 mg CaCl₂-2H₂O, 2 mg ZnSO₄-7H₂O, 2 mg MnSO4—H₂O, 50 mg L-tryptophan, 50 mg thiamine (vitamin B1), 50 mg nicotinic acid, and 4×10⁻⁵% biotin (10 microliters of a 20 mg per 50 mL solution). To these presterilized components, 800 mls of sterile water is added, optionally pre-warmed water (e.g., to 37° C.) to speed up dissolution of mixture. One can then swirl the culture vessel (e.g. bottle) and warm in water bath until the components are dissolved or a sterilized magnetic stir bar can be used to mix the components within the water. Once dissolved, appropriate labeled or unlabeled carbon and nitrogen sources can be added. For example, for ¹⁵N labeling, one can add about 10 grams of labeled ammonium sulfate or ammonium chloride. For ¹³C labeling, one can, for example, about 10 grams of labeled dextrose or about 20 grams of labeled glycerol.

The subject invention provides methods for culturing a cell using the growth or expression media disclosed herein, as well as compositions comprising cells in combination with the growth or expression medium. In certain embodiments, a culture medium as described herein is used to culture cells according to standard cell culture techniques that are well-known to one of ordinary skill in the art. In such techniques, the cells to be cultured are contacted with the medium of the invention under conditions favoring the cultivation of the cells (such as controlled temperature, humidity, lighting and atmospheric conditions). In various aspects of the invention, a culture medium is provided that can be used for the growth of yeast.

In one aspect of the invention, a culture medium is provided for the growth of a yeast host cell. Examples of yeast host cells cultured in accordance with the subject invention include, but are not limited to: Candida, Hansenula, Kluyveromyces, Pichia, Saccharomyces, Schizosaccharomyces, or Yarrowia cells. Various embodiments within this aspect of the invention include a yeast host cell selected from Saccharomyces carisbergensis, Saccharomyces cerevisiae, Saccharomyces diastaticus, Saccharomyces douglasii, Saccharomyces kluyveri, Saccharomyces norbensis, Saccharomyces oviformis, Yarrowia lipolytica, Hansenula polymorphs, Pichia pastoris or a Kluyveromyces lactis cell. Certain embodiments within this aspect of the invention include methylotrophic yeast cells.

In one embodiment, a culture medium including the components listed in Table 2 are used for the culture of yeast cells, such as Pichia pastoris, in their growth phase.

TABLE 2
Growth Phase Medium
1 L total volume

| | | |
|---|---|---|
| H2O | 800 | ml |
| adenine | 1.50 | g |
| guanosine | 1.95 | g |
| thymine | 0.60 | g |
| uracil | 1.50 | g |
| cytosine | 0.60 | g |
| sodium acetate | 4.50 | g |
| succinic acid | 1.50 | g |
| KH₂PO₄ | 10.88 | g |
| K₂HPO₄ | 2.12 | g |
| KOH | 0.68 | g |

post-autoclaving

| | | |
|---|---|---|
| 10x YNB | 100 | ml |
| 10x Dex | 100 | ml |
| Ampicillin | 250 | μL |
| metal/vitamin solution | 10 | ml |

With this embodiment, 10 mL per 1 L of a filter-sterilized metal/vitamin solution contains: 25 mg MgSO₄-7H₂O; 28 mg FeCl₃-6H₂O; 2 mg CaCl₂-2H₂O; 2 mg ZnSO₄-7H₂O; 2 mg MnSO₄—H₂O; 50 mg L-tryptophan; 50 mg thiamine (vitamin B1); 50 mg nicotinic acid (niacin); and 4×10⁻⁵% biotin (10.5 μl of 1000× Biotin per 10 ml). The 10× YNB (1 L) contains: 34 g YNB without amino acids and without ammonium sulfate; 100 g Ammonium sulfate; water to 1 L. The 10× Dex (1 L) contains: 200 g Dextrose and water to 1 L. The 1000× Biotin contains: 20 mg Biotin and 50 ml water.

Another embodiment provides an expression phase medium for yeast cells, including Pichia pastoris, as set forth in Table 3.

TABLE 3
Expression Phase Medium
250 ml

| | | |
|---|---|---|
| H2O | 225 | ml |
| Adenine | 0.38 | g |
| Guanosine | 0.49 | g |
| Thymine | 0.15 | g |
| Uracil | 0.38 | g |
| Cytosine | 0.15 | g |
| sodium acetate | 1.13 | g |
| succinic acid | 0.38 | g |
| KH₂PO₄ | 2.72 | g |
| K₂HPO₄ | 0.53 | g |
| KOH | 0.17 | g |

post-autoclaving

| | | |
|---|---|---|
| 10x YNB | 25 | ml |
| Methanol | 1.25 | ml |
| Ampicillin | 200 | μL |
| metal/vitamin solution | 2.5 | ml |

With this embodiment, 10 mL per 1 L of a filter-sterilized metal/vitamin solution contains: 25 mg MgSO₄-7H₂O; 28 mg FeCl₃-6H₂O; 2 mg CaCl₂-2H₂O; 2 mg ZnSO₄-7H₂O; 2 mg MnSO₄—H₂O; 50 mg L-tryptophan; 50 mg thiamine (vitamin B1); 50 mg nicotinic acid (niacin); and 4×10⁻⁵% biotin (10.5 μl of 1000× Biotin). The 10× YNB (1 L) contains: 34 g YNB without amino acids and without ammonium sulfate; 100 g Ammonium sulfate; and 1 L water. The 1000× Biotin contains: 20 mg Biotin and 50 ml water.

Another embodiment provides a medium for growing yeast cells, including Pichia pastoris, as set forth in Table 4.

TABLE 4
Reagent

| | quantity per liter media | |
|---|---|---|
| adenine (gm) | 1.50 | g |
| guanosine (gm) | 1.95 | g |
| thymine (gm) | 0.60 | g |
| uracil (gm) | 1.50 | g |
| cytosine (gm) | 0.60 | g |
| sodium acetate trihydrate (gm) | 4.50 | g |
| succinic acid (gm) | 1.50 | g |
| KH2PO4 (gm) | 10.88 | g |
| K2HPO4 (gm) | 2.12 | g |
| KOH (gm) | 0.68 | g |
| YNB w/o amino acids and | 3.40 | g |
| ammonium sulfate (gm) | | |
| methanol (ml) | 5 | g |
| dextrose (gm) | 20 | g |
| ammonium sulfate (gm) | 10 | g |
| MgSO4•7H2O (gm) | 0.025 | g |
| FeCl3•6H2O (gm) | 0.028 | g |
| CaCl2•2H2O (gm) | 0.02 | g |
| ZnSO4•7H2O (gm) | 0.02 | g |
| MnSO4•H2O (gm) | 0.02 | g |
| L-tryptophan (gm) | 0.05 | g |
| thiamine (vitamin B1) (gm) | 0.05 | g |
| nicotinic acid (niacin) (gm) | 0.05 | g |
| biotin (gm) | 0.0000042 | g |

In various embodiments the any liquid media disclosed herein (e.g., the media disclosed in Tables 2, 3 or 4) can also be further diluted from its original concentration of ingredients/components. For example, any liquid medium can be diluted between about 2 and about 20 (or more) fold, e.g., about 2, 3, 4, 5, 6, 7, 8, 9, 10, 11, 12, 13, 14, 15, 16, 17, 18, 19, 20 (or more) fold. In certain preferred embodiments, the medium can be diluted 2, 5 or 10 fold. Other embodiments provide for the alteration of the amounts of the components listed within any one of Tables 2, 3 or 4 (including any component substituted for succinate and/or succinic acid) by up to 5%, 10%, 15%, 20%, 25%, 30%, 35%, 40%, 45%, or 50% (e.g., any amount listed within the table can be altered such that its value is ±50% (for example, adenine can have its amount listed as 1.50 g±50%)).

Yet another embodiment of the invention provides a dry medium to which sterile water can be added to provide a growth medium preparation. In this embodiment, a container comprising about 10.88 g KH₂PO₄, about 2.12 g K₂HPO₄, about 0.68 g of KOH, about 4.5 g sodium acetate (trihydrate), about 1.5 g adenine, about 1.95 g guanosine, about 0.60 g thymine, about 1.5 g uracil, about 0.60 g cytosine, and about 1.5 g succinic acid. 800 mls of sterile, optionally endotoxin free, water can be added to the dry medium and the dry medium solubilized, optionally in pre-warmed water at 37° C. to speed the dissolving process. Once dissolved 100 mls of sterilized yeast nitrogen base (YNB), prepared following the manufacturers' recommendations, can be added as can 100 ml of sterilized 10× dextrose solution (200 gm/L), 10 ml of sterile metal-vitamin solution, and, optionally, appropriate antibiotics (such as ampicillin). The metal-vitamin solution can be sterilized according to methods known in the art and 10 mls contains 25 mg MgSO₄-7H₂O, 28 mg FeCl₃-6H₂O, 2mg CaCl₂-2H₂O, 2 mg ZnSO₄-7H₂O, 2 mg MnSO₄—H₂O, 50 mg L-tryptophan, 50 mg thiamine (vitamin B1), 50 mg nicotinic acid, and 4×10⁻⁵% biotin (10 microliters of a 1000× biotin solution; 20 mg biotin in a 50 mL solution). In several additional embodiments, the sterilized yeast nitrogen base, metal-vitamin components, antibiotic and/or biotin can be added in dry form (e.g., from sterile lyophilized sources).

The culture media disclosed herein are particularly useful for producing recombinant proteins. In this aspect of the invention, a yeast cell transformed with a nucleic acid construct encoding a desired recombinant protein, such as a transformed Pichia pastoris cell, is cultured in a growth phase medium and transitioned to an expression phase medium in order to produce a desired recombinant protein. Examples of recombinant proteins that can be produced in accordance with the subject invention include, but are not limited to:

a) addressins;

b) antibodies (e.g., full size immunoglobulins, Fab fragments, scFvs, bispecific Fvs, minibodies, single variable domains, antibody fusion proteins, monoclonal antibodies or diabodies;

c) anti-sepsis proteins (such as bactericidal permeability-increasing protein);

d) autoantigens;

e) binding proteins (steroid binding proteins, growth hormone or growth factor binding proteins, interleukin binding proteins, CSF binding proteins, interferon receptors and the like; G-coupled receptors);

f) blood factors/proteins, hormones or interleukins (such as thrombin, serum albumin, Factor VII, Factor VIII, insulin, Factor IX, Factor X, tissue plasminogen activator, Protein C, von Wilebrand factor, antithrombin III, glucocerebrosidase, epidermal growth factor, lactoferrin, erythropoietin, granulocyte colony stimulating factor (G-CSF), granulocyte-macrophage colony stimulating factor (GM-CSF) or modified Factor VIII anticoagulants such as huridin), calcitonin, IL-1, IL-2, IL-3, IL-4, IL-5, IL-6, IL-7, IL-8, IL-9, IL-10, IL-11, IL-15, Il-16, Il-18, IL-23, IL-24, M-CSF, platelet derived growth factor (PDGF), MSF, FLT-3 ligand, EGF, fibroblast growth factor (FGF; e.g., FGF-1, FGF-2, FGF-3, FGF-4, FGF-5, FGF-6, or FGF-7), insulin-like growth factors (e.g., IGF-1, IGF-2); vascular endothelial growth factor (VEGF); interferons (e.g., IFN-γ, IFN-α, IFN-β); leukemia inhibitory factor (LIF); ciliary neurotrophic factor (CNTF); oncostatin M; stem cell factor (SCF); nerve growth factor; transforming growth factors (e.g., TGF-α, TGF-β1, TGF-β2, TGF-β3), TNF-α, TNF-β or chemokines (such as, but not limited to, BCA-1/BLC-1, BRAK/Kec, CXCL16, CXCR3, ENA-78/LIX, Eotaxin-1, Eotaxin-2/MPIF-2, Exodus-2/SLC, Fractalkine/Neurotactin, GROalpha/MGSA, HCC-1, I-TAC, Lymphotactin/ATAC/SCM, MCP-1/MCAF, MCP-3, MCP-4, MDC/STCP-1, ABCD-1, MIP-1α, MIP-1β, MIP-2α/GROβ, MIP-3α/Exodus/LARC, MIP-3β/Exodus-3/ELC, MIP-4/PARC/DC-CK1, PF-4, RANTES, SDF 1α, TARC, or TECK), hormones, such as insulin, thyroid hormone, catecholamines, gonadotrophins (e.g., human chorionic gonadotropin (HCG)), trophic hormones, prolactin, oxytocin, bovine somatotropin, leptins and the like);

g) enzymes (such as streptokinase, cholesterol biosynthestic or degradative enzymes, steriodogenic enzymes, kinases, phosphodiesterases, methylases, de-methylases, dehydrogenases, cellulases, proteases, lipases, phospholipases, aromatases, cytochromes, adenylate or guanylaste cyclases, neuramidases, N-acetyl neuraminic acid (Neu5Ac) synthase or Neu5Ac lyase, epimerase, CMP-Neu5 Ac synthase, a CMP- Neu5Ac transporter, sialyltransferase, FLP recombinase;

h) autoimmune, bacterial, viral, parasitic or cancer antigens, such as colorectal cancer antigen, G protein of Respiratory Syncytial Virus, gastrointestinal cancer antigen, hepatitis B surface antigen, human papilloma virus antigens (such as E6 protein, E7 protein, fragments, variants or fusions thereof), influenza antigen polypeptides (such as hemagglutinin (HA), neuraminidase (NA), membrane ion channel M2 (M2), matrix protein M1 (M1), a portion of hemagglutinin (HA), a portion of neuraminidase (NA), a portion of membrane ion channel (M2), a portion of matrix protein M1 (M1), fusion proteins thereof, immunogenic portions or combinations thereof), malaria antigens (such as, Apical Membrane Antigen-1 (AMA-1), Merozoite Surface Antigen-1 (MSP-1), circumsporozoite protein (CSP), saponin, Norovirus and Sapovirus antigens, Norwalk virus capsid protein, oncoproteins or proto-oncoproteins (e.g., cell cycle proteins), parasitic antigens, rabies virus glycoprotein, rabies virus nucleoprotein, Yersinia pestis antigens: anti-phagocytic capsular envelope glycoprotein (Fl) and the low calcium-response V (LcrV), or any immunogenic protein or peptide capable of eliciting an immune response against Yersinia Pestis, anthrax antigens (such as protective antigen (PA), lethal factor (LF) and edema factor (EF) and fragments thereof, a portion of protective antigen (PA), a portion of lethal factor (LF) and a portion of edema factor (EF));

i) muscle proteins (such as myosin or tropomyosin and the like);

j) soluble major histocompatibility complex antigens;

k) structural proteins (such as collagen, fibroin, fibrinogen, elastin, tubulin, actin, and myosin) or integral membrane proteins (such as receptors (e.g., G-coupled receptors), porins, or transporters (such as glucose, norepinephrine or serotonin transporters));

l) fusion proteins, such as a CTS domain of N-acetylglucosaminyl transferase (GNTl) fused to a catalytic domain of beta-1,4 galactosyltransferase (GaIT);

m) selectins;

n) T cell receptors;

o) transcription and translation factors;

p) tumor growth suppressing proteins (e.g., angiostatin or endostatin);

q) myeloproteins; and

r) neuroactive proteins.

All patents, patent applications, provisional applications, and publications referred to or cited herein are incorporated by reference in their entirety, including all figures and tables, to the extent they are not inconsistent with the explicit teachings of this specification.

Following are examples which illustrate procedures for practicing the invention. These examples should not be construed as limiting. All percentages are by weight and all solvent mixture proportions are by volume unless otherwise noted.

### EXAMPLE 1

### Expression Medium Preparation Protocol A: Full Autoclaving

Add 225 mls of endotoxin free water to an autoclaved bottle. Then add 2.72 g KH₂PO₄, 0.53 g K₂HPO₄, and 0.17 g of KOH. Next add 1.13 g sodium acetate (trihydrate), 0.38 g adenine, 0.49 g guanosine, 0.15 g thymine, 0.38 g uracil, 0.15 g cytosine, and 0.38 g succinic acid.

Place the bottle in an autoclave bin filled with water, and autoclave at 250° F. for 20 minutes. Swirl bottle immediately upon removal from autoclave to dissolve any residual reagents.

Allow to cool to room temperature, and add 25 mls of filter sterilized yeast nitrogen base (YNB) prepared following the manufactures recommendations, 1.25 ml anhydrous methanol, 2.5 ml of the metal-vitamin solution, and an appropriate antibiotics. The metal-vitamin solution is filter sterilized and 10 mls contains 25 mg MgSO₄-7H₂O, 28 mg FeCl₃-6H₂O, 2 mg CaCl₂-2H₂O, 2 mg ZnSO₄-7H₂O, 2 mg MnSO₄-H₂O, 50 mg L-tryptophan, 50 mg thiamine (vitamin B1), 50 mg nicotinic acid, and 4×10⁻⁵% biotin.

### EXAMPLE 2

### Expression Medium Preparation Protocol B: Two Part Autoclaving

Add 150 mls of endotoxin free water to an autoclaved bottle. Then add 2.72 g KH₂PO₄, 0.53 g K₂HPO₄, and 0.17 g of KOH.

In a separate bottle prepare the nucleotide solution by adding 75 mls of endotoxin free water then add 1.13 g sodium acetate (trihydrate), 0.38 g adenine, 0.49 g guanosine, 0.15 g thymine, 0.38 g uracil, 0.15 g cytosine, and 0.38 g succinic acid.

Place the bottle containing the nucleotide solution in an autoclave bin filled with enough water to be higher than the liquid in the nucleotide solution bottle. Autoclave both bottles at 250° F. for 20 minutes. Swirl both bottles immediately upon removal from autoclave to dissolve any residual reagents.

Allow to cool to room temperature, and add 25 mls of filter sterilized yeast nitrogen base (YNB) prepared following the manufactures recommendations, 1.25 ml anhydrous methanol, 2.5 ml of the metal-vitamin solution, and an appropriate antibiotics. The metal-vitamin solution is filter sterilized and 10 mls contains 25 mg MgSO₄-7H₂O, 28 mg FeCl₃-6H₂O, 2 mg CaCl₂-2H₂O, 2 mg ZnSO₄-7H₂O, 2 mg MnSO₄ -H₂O, 50 mg L-tryptophan, 50 mg thiamine (vitamin B1), 50 mg nicotinic acid, and 4×10⁻⁵% biotin.

### EXAMPLE 3

### Expression Medium Preparation Protocol C: Dry Reagents, No Autoclaving

In a sterile bottle add 2.72 g KH₂PO₄, 0.53 g K₂HPO₄, and 0.17 g of KOH. Next add 1.13 g sodium acetate (trihydrate), 0.38 g adenine, 0.49 g guanosine, 0.15 g thymine, 0.38 g uracil, 0.15 g cytosine, and 0.38 g succinic acid.

Add 225 mls of sterilized endotoxin free water to the reagent mixture, pre-warming water to 37° C. will speed dissolving process. Swirl bottle periodically till dissolved or add a sterilized magnetic stir bar and allow to stir at a medium speed.

Once dissolved add 25 mls of filter sterilized yeast nitrogen base (YNB) prepared following the manufactures recommendations, 1.25 ml anhydrous methanol, 2.5 ml of the metal-vitamin solution, and an appropriate antibiotics. The metal-vitamin solution is filter sterilized and 10 mls contains 25 mg MgSO₄-7H₂O, 28 mg FeCl₃-6H₂O, 2 mg CaCl₂-2H₂O, 2 mg ZnSO₄-7H₂O, 2 mg MnSO₄ -H₂O, 50 mg tryptophan, 50 mg thiamine (vitamin B1), 50 mg nicotinic acid, and 4×10⁻⁵% biotin.

### EXAMPLE 4

### Growth Medium Preparation Protocol A: Full Autoclaving

Add 800 mls of endotoxin free water to an autoclaved bottle. Then add 10.88 g KH₂PO₄, 2.12 g K₂HPO₄, and 0.68 g of KOH. Next add 4.5 g sodium acetate (trihydrate), 1.5 g adenine, 1.95 g guanosine, 0.60 g thymine, 1.5 g uracil, 0.60 g cytosine, and 1.5 g succinic acid. Prepare a 100 ml of 10× dextrose solution, 200 g dextrose per 1 L water, in a separate bottle.

Place the bottle containing the nucleotides in an autoclave bin filled with water, and autoclave the media and the 10× dextrose solution at 250° F. for 20 minutes. Swirl both bottles upon removal from autoclave to dissolve any residual reagents.

Allow to cool to room temperature, and add 100 mls of filter sterilized yeast nitrogen base (YNB) prepared following the manufactures recommendations, 100 ml of the 10× dextrose, 10 ml of the metal-vitamin solution, and an appropriate antibiotics. The metal-vitamin solution is filter sterilized and 10 mls contains 25 mg MgSO₄-7H₂O, 28 mg FeCl₃-6H₂O, 2 mg CaCl₂-2H₂O, 2 mg ZnSO₄-7H₂O, 2 mg MnSO₄—H₂O, 50 mg L-tryptophan, 50 mg thiamine (vitamin B1), 50 mg nicotinic acid, and 4×10⁻⁵% biotin.

### EXAMPLE 5

### Growth Medium Preparation Protocol B: Dry Reagents, No Autoclaving

In a sterile bottle add 10.88 g KH₂PO₄, 2.12 g K₂HPO₄, and 0.68 g of KOH. Next add 4.5 g sodium acetate (trihydrate), 1.5 g adenine, 1.95 g guanosine, 0.60 g thymine, 1.5 g uracil, 0.60 g cytosine, and 1.5 g succinic acid.

Add 800 mls of endotoxin free water to the reagent mixture, pre-warming water to 37° C. will speed dissolving process. Swirl bottle and warm in water bath till dissolved or add a sterilized magnetic stir bar and allow to stir at a medium speed.

Once dissolved add 100 mls of filter sterilized yeast nitrogen base (YNB) prepared following the manufactures recommendations, 100 ml of filter sterilized 10× dextrose solution (200 gm/L), as well as 10 ml of metal-vitamin solution, and appropriate antibiotics. The metal-vitamin solution is filter sterilized and 10 mls contains 25 mg MgSO₄-7H₂O, 28 mg FeCl₃-6H₂O, 2 mg CaCl₂-2H₂O, 2 mg ZnSO₄-7H₂O, 2 mg MnSO₄—H₂O, 50 mg L-tryptophan, 50 mg thiamine (vitamin B1), 50 mg nicotinic acid, and 4×10⁻⁵% biotin.

### EXAMPLE 6

### Comparison of Different Protein Expression in Pichia Pastoris

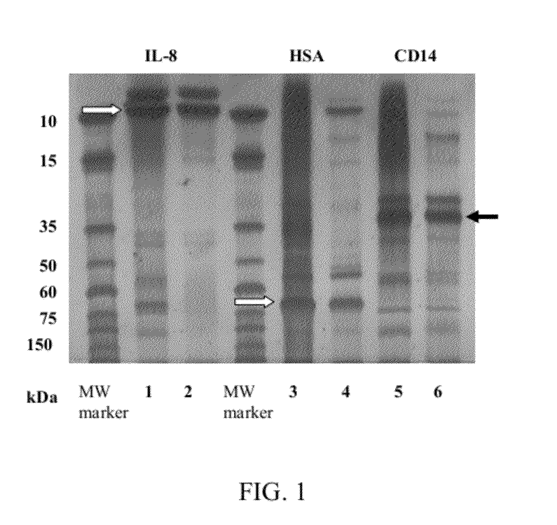

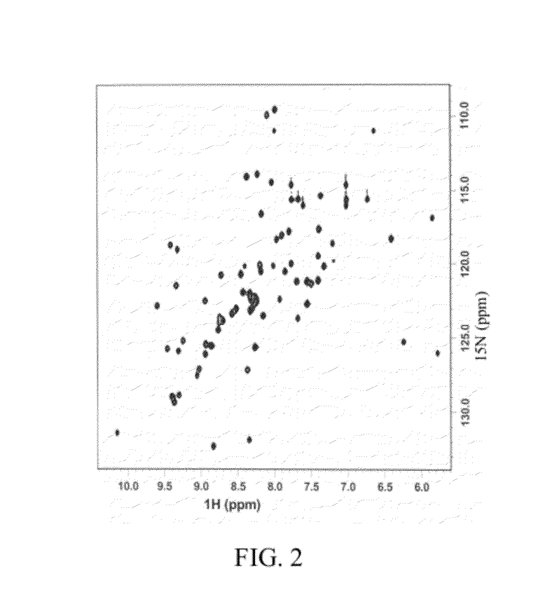

Three different proteins, Interleukin-8 (IL-8), Human Serum Albumin (HSA) and glycoprotein CD14 were grown and secreted by transformed Pichia pastoris cells in 50 mL shake-flask cultures of rich BMMY media and formulation media of the subject invention under identical conditions. The experimental protocol for preparing the formulation media for growth and expression are as described above in examples 1 and 4. SDS-PAGE gels were run on each secreted sample without purification (FIG. 1). The bands for the respective proteins are marked with arrows on the gel. Total yields of protein in the 50 mL shake-flask cultures are comparable in the BMMY media and formulation media of the subject invention and ranged from 0.5-2 mg. The yields in fermentation cultures ranged from 3-10 mg (data not shown). The formulation media of the subject invention is much cleaner for secreted proteins as seen from the gel, easing purification. For isotopic labeling of IL-8 with 15N, the unlabeled ammonium sulfate in the formulation media was replaced with 15N ammonium sulfate. Expressed IL-8 in formulation media of the subject invention was completely labeled with 15N, as seen in FIG. 2.

It should be understood that the examples and embodiments described herein are for illustrative purposes only and that various modifications or changes in light thereof will be suggested to persons skilled in the art and are to be included within the spirit and purview of this application. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

We claim:

1. A medium comprising:
a) adenine, guanosine, thymine, uracil, cytosine, sodium acetate, KH₂PO₄, K₂HPO₄, KOH, 10× YNB, a metal/vitamin solution and one or more of the following compounds succinic acid/succinate, pyruvate, oxaloacetate or alphaketoglutarate;
b) a medium as set forth in Table 2;
c) a medium as set forth in Table 3; or
d) a medium as set forth in Table 4.

2. The medium according to claim 1, further comprising dextrose or methanol.

3. The medium according to claim 1, wherein the medium is a liquid.

4. The medium according to claim 1, wherein the medium is a dry powder.

5. The medium according to claim 1, wherein said medium also contains an antibiotic.

6. The medium according to claim 5, wherein said antibiotic is ampicillin.

7. The medium according to claim 1, wherein said medium comprises one or more of the following compounds: labeled or unlabeled succinate; labeled or unlabeled acetate; labeled or unlabeled ammonium chloride; labeled or unlabeled glycerol or labeled or unlabeled ammonium sulfate.

8. The medium according to claim 1, wherein said medium comprises a formulation according to Table 2 or Table 3 or Table 4.

9. The medium according to claim 1, wherein said medium is a dry medium composition comprising about 10.88 g KH₂PO₄, about 2.12 g K₂HPO₄, about 0.68 g of KOH, about 4.5 g sodium acetate (trihydrate), about 1.5 g adenine, about 1.95 g guanosine, about 0.60 g thymine, about 1.5 g uracil, about 0.60 g cytosine, and about 1.5 g succinic acid.

10. The dry medium composition according to claim 9, wherein said dry medium composition is sterilized.

11. The dry medium composition according to claim 9, wherein said dry medium composition is provided within a container, said container being optionally sterilized.

12. The dry medium according to claim 9, said thy medium further comprising about 25 mg MgSO₄-7H₂O, about 28 mg FeCl₃-6H₂O, about 2 mg CaCl₂-2H₂O, about 2 mg ZnSO₄-7H₂O, about 2 mg MnSO₄—H₂O, about 50 mg L-tryptophan, about 50 mg thiamine (vitamin B1), about 50 mg nicotinic acid and about 0.004 mg biotin.

13. The medium composition according to claim 1, wherein said medium composition is a dry medium composition comprising 10.88 g KH₂PO₄, 2.12 g K₂HPO₄, 0.68 g of KOH, 4.5 g sodium acetate (trihydrate), 1.5 g adenine, 1.95 g guanosine, 0.60 g thymine, 1.5 g uracil, 0.60 g cytosine, and 1.5 g succinic acid.

14. The dry medium composition according to claim 13, wherein said dry medium composition is sterilized.

15. The dry medium composition according to claim 13, wherein said dry medium composition is provided within a container, said container being optionally sterilized.

16. The dry medium according to claim 13, said dry medium further comprising 25 mg MgSO₄-7H₂O, 28 mg FeCl₃-6H₂O, 2 mg CaCl₂-2H₂O, 2 mg ZnSO₄-7H₂O, 2 mg MnSO₄—H₂O, 50 mg L-tryptophan, 50 mg thiamine (vitamin B1), 50 mg nicotinic acid and 0.004 mg biotin,

17. A process for producing a medium according to claim 1, comprising:
(a) combining KH₂PO₄, K₂HPO₄, KOH, sodium acetate, adenine, guanosine, thymine, uracil, cytosine, and succinic acid/succinate with water;
(b) autoclaving the ingredients of step (a) and then allowing to cool to room temperature; and
(c) adding to the cooled ingredients of step (b) sterilized YNB, anhydrous methanol, metal-vitamin solution, and one or more antibiotic.

18. The process according to claim 17, wherein the KH₂PO₄, K₂HPO₄, and KOH of step (a) are combined in a first vessel with water and the sodium acetate, adenine, guanosine, thymine, uracil, cytosine, and succinic acid of step (a) are combined in a second vessel with water.

19. The process according to claim 17, wherein the metal-vitamin solution comprises: MgSO₄-7H₂O, FeCl₃-6H₂O, CaCl₂-2H₂O, ZnSO₄-7H₂O, MnSO₄—H₂O, L-tryptophan, thiamine (vitamin B1), nicotinic acid (niacin), and about 0.004 mg biotin.

20. The process according to claim 17, wherein the antibiotic is ampicillin.

21. The process according to claim 17, wherein one or more component selected from labeled or unlabeled ammonium chloride; labeled or unlabeled glycerol; or labeled or unlabeled ammonium sulfate; or labeled or unlabeled dextrose is added during formulation of said medium and said component is sterile or sterilized during the preparation of said medium.

22. A method of culturing cells in a medium comprising contacting the cells with the medium according to claim 1.

23. The method according to claim 22, wherein the cells are yeast.

24. The method according to claim 23, wherein the yeast are methylotrophic yeast.

25. The method according to claim 24, wherein the yeast is Pichia pastoris.

26. A composition comprising a medium according to claim 1 and a yeast cell.

27. The composition according to claim 26, wherein the yeast is Pichia pastoris.

28. A method for producing recombinant proteins comprising culturing a recombinant protein producing yeastin a medium according to claim 1 under conditions that allow for the expression of said recombinant protein.

29. The method according to claim 28, wherein the recombinant protein is selected from the group consisting of:
a) addressins;
b) antibodies;
c) anti-sepsis proteins;
d) autoantigens;
e) binding proteins;
f) blood factors/proteins, hormones or interleukins;
g) enzymes;
h) autoimmune, bacterial, viral, parasitic or cancer antigens, such as colorectal cancer antigen, G protein of Respiratory Syncytial Virus, gastrointestinal cancer antigen, hepatitis B surface antigen, human papilloma virus antigens;
i) muscle proteins;
j) soluble major histocompatibility complex antigens;
k) structural proteins;
l) fusion proteins;
m) selectins;
n) T cell receptors;
o) transcription and translation factors;
p) tumor growth suppressing proteins;
q) myeloproteins; and
r) neuroactive proteins.

30. The method according to claim 29, wherein said yeast is cultured in a medium that is endotoxin free and an endotoxin free recombinant protein is produced.

31. The method according to claim 28, wherein the protein is selected from the group consisting of interleukin-8, human serum albumin, and glycoprotein CD 14.

32. The method according to claim 28, wherein said medium comprises a labeled source of carbon and/or a labeled source of nitrogen.

33. The method according to claim 32, wherein said labeled source of carbon and/or nitrogen is selected from ¹⁵N labeled ammonium sulfate or ammonium chloride and/or ¹³C labeled dextrose or glycerol.

34. A medium comprising:
a) succinate and/or succinic acid, said succinate and/or succinic acid being optionally substituted with pyruvate, oxaloacetate or alphaketoglutarate, or any combination thereof;
b) a source of acetate selected from: 1) sodium acetate; 2) potassium acetate; 3) acetic acid; 4) sodium acetate and potassium acetate; 5) sodium acetate and acetic acid; 6) potassium acetate and acetic acid; or 6) sodium acetate, potassium acetate and acetic acid; and, optionally,
c) one or more of the following components: adenine, guanosine, thymine, uracil, cytosine, KH₂PO₄, K₂HPO₄, KOH, 10× YNB, an antibiotic and/or metal/vitamin solution/mixture as disclosed herein.

35. The medium according to claim 34, wherein said medium comprises pyruvate, oxaloacetate or alphaketoglutarate, or any combination thereof, substituted for the succinate and/or succinic acid listed in subpart (a).

36. The medium according to claim 34, wherein said medium contains components in amounts as set forth in any one of Tables 2, 3 or 4, provided that if the medium contains components that are to be substituted for succinate and/or succinic acid, the total amount of the substituted components equals the amount of succinate and/or succinic acid that is present in a medium as set forth in Table 2, 3 or 4.

37. The medium according to claim 36, wherein the amount of any component is altered by up to ±50%.
